(19) United States
(12) Patent Application Publication
(10) Pub. No.: US 2006/0206160 A1
(43) Pub. Date: Sep. 14, 2006

(54) Process and electrostimulation device for treating obesity and/or gastroesophageal reflux disease

(75) Inventors: Valerio Cigaina, Villorba (IT); Claudia Knippig, Magdeburg (DE)

Correspondence Address: FITCH EVEN TABIN AND FLANNERY, 120 SOUTH LA SALLE STREET, SUITE 1600, CHICAGO, IL, 60603-3406, US

(73) Assignee: Transneuronix, Inc.

(21) Appl. No.: 10/154,012

(22) Filed: May 23, 2002

Related U.S. Application Data
(63) Continuation-in-part of application No. 09/713,556, filed on Nov. 15, 2000, now Pat. No. 6,615,084.

(51) Int. Cl. A61N 1/00

(52) U.S. Cl. 607/40

(57) ABSTRACT

An improved process using electrostimulation for treating obesity and/or related motor disorders is provided. The improved method of this invention provides electrostimulation on the lesser curvature of the stomach. Preferably, the electrostimulation or pacemaker device provides electrostimulation to the lower or distal end of the lesser curvature (i.e., towards the pylorus) for the treatment or control of obesity. Preferably, the electrostimulation or pacemaker device provides electrostimulation to the upper or proximal end of the lesser curvature for the treatment or control of gastroesophageal reflux disease. In one embodiment, the process employs stimulation of the lesser curvature at a rate of about 2 to about 14 pulses/minute with each pulse lasting about 0.5 to about 4 seconds such that there is a pause of about 3 to about 30 seconds between the pulses.

### RELATED APPLICATION

The present application is a continuation-in-part of U.S. patent application Ser. No. 09/713,556, filed on Nov. 15, 2000, and which is hereby incorporated by reference.

### FIELD OF THE INVENTION

The present invention relates to an improved process using electrostimulation for treating obesity, especially morbid obesity, gastroesophageal reflux disease (GERD), and other syndromes related to motor disorders of the stomach. The improved method of this invention provides electrostimulation on the lesser curvature of the stomach which provides improved control of obesity, improved treatment of gastroesophageal reflux disease, and other syndromes related to motor disorders of the stomach.

### BACKGROUND OF THE INVENTION

The modern surgical orientation with regard to obesity generally entails the reduction of gastric compliance, with the aim of limiting the subject's ability to ingest food, or of reducing the food absorption surface by shortening or bypassing part of the digestive canal; both aims are sought in some surgical procedures. Until recently, surgery was the only therapy that ensures real results in patients who have exceeded obesity values close to or greater than about 40 BMI (ratio of weight in kilograms to the square of the height in meters).

All of the major surgical procedures (e.g., removal or blocking off of a portion of the stomach) currently in use have some immediate and/or delayed risks. Thus, surgery is usually considered as an extreme solution when all less invasive procedures fail. Furthermore, even surgical treatment fails in some cases, thereby requiring the surgeon to restore the original anatomical situation.

More recently, methods have been successfully employed whereby an electrostimulation device is implanted on the stomach wall. For example, U.S. Pat. No. 5,423,872 (Jun. 13, 1995) provided a process for the treatment of obesity and related disorder employing an electrostimulator or pacemaker attached to the antrum or greater curvature of the stomach. U.S. Pat. No. 5,690,691 (Nov. 25, 1997) provided a portable or implantable gastric pacemaker including multiple electrodes positionable on the inner or outer surface of an organ in the gastrointestinal tract which are individually programmed to deliver a phased electrical stimulation to pace peristaltic movement of material through the gastrointestinal tract. Although these methods have generally been successful, it is still desirable to provide improved methods for such treatments. The present invention provides such an improved process.

Gastroespohageal reflux disease or GERD is a digestive disorder affecting the lower esophageal sphincter whereby stomach contents are allowed to flow back into the esophagus. In normal patients, the lower esophageal sphincter allows food to pass from the esophagus into the stomach while preventing food and the generally acidic stomach digestive juices from flowing back into the esophagus. If the lower esophageal sphincter is weak or does not relax and/or contract appropriately, the contents of the stomach (i.e., acid reflux) may enter into the esophagus causing so-called heartburn and, in some cases, damage to the lining of the esophagus.

The majority of cases of gastroespohageal reflux disease can be managed with reasonable success with lifestyle changes and the use of conventional antacids. If such conservative measures fail, however, more aggressive drug treatment or surgery may be appropriate. Especially, as minimally invasive surgical procedures have become more common, surgical methods are playing increased roles in the treatment of gastroespohageal reflux disease and especially chronic gastroespohageal reflux disease.

U.S. Pat. No. 5,716,385 (Feb. 10, 1998) provided a method for treatment of gastroespohageal reflux disease using electrical stimulation of the skeletal muscles of the crural diaphragm, thereby indirectly causing contraction of the lower esophageal sphincter.

U.S. Pat. No. 6,077,984 (Aug. 1, 2000) provides a method for the direct stimulation of the lower esophageal sphincter for treatment of gastroespohageal reflux disease. This method employs an electrode on or around the lower esophageal sphincter at the gastro-esophageal junctnion and a sensor affixed to the esophageal wall to sense normal episodes of motility (i.e., swallowing). Generally the electrode is located below the diaphragm (i.e., within the abdominal cavity and inferior to the diaphragm) and the sensor above the diaphragm (i.e., within the thoracic cavity and superior to the diaphragm) with a lead providing electrical connection between the electrode and sensor which tunnels through the diaphragm.

It is desirable to provide improved methods for the treatment of gastroesophageal reflux disease. The present invention provides such improved methods.

### SUMMARY OF THE INVENTION

The present invention provides a process for treating obesity and/or related motor disorders by providing an electrostimulation or pacemaker device attached to the lesser curvature of the stomach. Preferably, the electrostimulation or pacemaker device provides electrostimulation to the lower or distal end of the lesser curvature (i.e., towards the pylorus) for the treatment or control of obesity. The present invention also provides a process for treating gastroesophageal reflux disease by providing an electrostimulation or pacemaker device attached to the lesser curvature of the stomach. Preferably, the electrostimulation or pacemaker device provides electrostimulation to the upper or proximal end of the lesser curvature for the treatment or control of gastroesophageal reflux disease.

Preferably, the processes of this invention employ stimulation of the lesser curvature at a rate of about 2 to about 14 pulses/minute with each pulse lasting about 0.5 to about 4 seconds such that there is a pause of about 3 to about 30 seconds between the pulses. More preferably, the pulse rate is about 12 pulses/minute with each pulse lasting about 2 seconds with a pause of about 3 seconds between pulses. Preferably, the pulse amplitude is about 0.5 to about 15 milliamps. More preferable, each pulse consists of a train of micro-bursts with a frequency of about 5 to about 100 sec⁻¹.

The process of the present invention involves treatment of obesity and other syndromes related to motor disorders of the stomach of a patient. The process comprises artificially altering, using sequential electrical pulses for preset periods of time, the natural gastric motility of the patient to prevent or slow down stomach emptying, thereby slowing food transit through the digestive system.

The present invention provides a method for treatment of a motor disorder of a patient's stomach, said method comprising implanting an electrostimulation device comprising one or more electrostimulation leads and an electrical connector for attachment to a pulse generator such that the one or more electrostimulation leads are attached to, or adjacent to, lesser curvature of the patient's stomach, whereby electrical stimulation can be provided to the lesser curvature through the one or more electrostimulation leads; and supplying electrical stimulation to the lesser curvature through the one or more electrostimulation leads.

This invention also provides a method for treatment of a motor disorder of a patient's stomach, said method comprising implanting an electrostimulation device comprising an elongated body with a proximal and a distal end and having one or more electrostimulation leads and an electrical connector for attachment to a pulse generator at the proximal end such that the one or more electrostimulation leads are attached to, or adjacent to, lesser curvature of the patient's stomach, whereby electrical stimulation can be provided to the lesser curvature through the one or more electrostimulation leads and whereby, once the electrostimulation device is implanted, the one or more electrostimulation leads are at the distal end of the elongated body; and supplying electrical stimulation to the lesser curvature through the one or more electrostimulation leads.

The process of the present invention also involves treatment of gastroesophageal reflux disease in a patient. The process comprises artificially altering, using sequential electrical pulses for preset periods of time, the natural gastric motility of the patient to provide electrostimulion of the lesser curvature of the stomach and thereby provide control to the lower esophageal sphincter.

The present invention provides a method for treatment of gastroesophageal reflux disease in a patient, said method comprising implanting an electrostimulation device comprising one or more electrostimulation leads and an electrical connector for attachment to a pulse generator such that the one or more electrostimulation leads are attached to, or adjacent to, lesser curvature of a patient's stomach, whereby electrical stimulation can be provided to the lesser curvature through the one or more electrostimulation leads; and supplying electrical stimulation to the lesser curvature through the one or more electrostimulation leads.

This invention also provides a method for treatment of gastroesophageal reflux disease in a patient, said method comprising implanting an electrostimulation device comprising an elongated body with a proximal and a distal end and having one or more electrostimulation leads and an electrical connector for attachment to a pulse generator at the proximal end such that the one or more electrostimulation leads are attached to, or adjacent to, lesser curvature of a patient's stomach, whereby electrical stimulation can be provided to the lesser curvature through the one or more electrostimulation leads and whereby, once the electrostimulation device is implanted, the one or more electrostimulation leads are at the distal end of the elongated body; and supplying electrical stimulation to the lesser curvature through the one or more electrostimulation leads.

### BRIEF DESCRIPTION OF THE DRAWING

FIG. 1 is a sectional view of a stomach showing the device of the invention in two places along the lesser curvature of the stomach. Device 42 is placed on the distal end of the lesser curvature which is the preferred location for treatment or control of obesity. Device 46 is placed on the proximal end of the lesser curvature which is the preferred location for treatment or control of gastroesophageal reflux disease. Generally, only one such device (i.e., 42 or 46) would be used for a particular patient with its preferred location depending on the condition to be treated.

FIG. 2 is a schematic representation (not to scale) of a preferred microburst pulse train provided to the lesser curvature of the stomach.

### DETAILED DESCRIPTION OF THE PREFERRED EMBODIMENTS

The present invention provides a process for treating obesity and/or related motor disorders by providing an electrostimulation or pacemaker device attached to the lesser curvature of the stomach. Preferably, the electrostimulation or pacemaker device provides electrostimulation to the distal end of the lesser curvature (i.e., towards the pylorus) for the treatment or control of obesity. The present invention also provides a process for treating gastroesophageal reflux disease or disorder by providing an electrostimulation or pacemaker device attached to the lesser curvature of the stomach. Preferably, the electrostimulation or pacemaker device provides electrostimulation to the upper or proximal end of the lesser curvature (i.e., towards the lower esophageal sphincter) for the treatment or control of gastroesophageal reflux disease.

Preferably, the processes of this invention employ stimulation of the lesser curvature at a rate of about 2 to about 14 pulses/minute with each pulse lasting about 0.5 to about 4 seconds such that there is a pause of about 3 to about 30 seconds between the pulses. More preferably, the pulse rate is about 12 pulses/minute with each pulse lasting about 2 seconds with a pause of about 3 seconds between pulses. Preferably, the pulse amplitude is about 0.5 to about 15 milliamps. More preferable, each pulse consists of a train of micro-bursts with a frequency of about 5 to about 100 sec⁻¹.

The process of the present invention involves treatment of obesity and other syndromes related to motor disorders of the stomach of a patient. The process comprises artificially altering, using sequential electrical pulses for preset periods of time, the natural gastric motility of the patient to prevent or slow down stomach emptying, thereby slowing food transit through the digestive system. It has been surprisingly found that placement of an electrostimulator on the lesser curvature, and even more preferably on the distal end of the lesser curvature near or adjacent to the angular notch, provides superior results in the control of obesity as compared to placement on the fundus, greater curvature, or antrum.

The process of the present invention also involves treatment of gastroesophageal reflux disease of a patient. The process comprises artificially altering, using sequential electrical pulses for preset periods of time, the lower esophageal sphincter to reduce or prevent return of the stomach's contents back up into the esophagus. It has been surprisingly found that placement of an electrostimulator on the lesser curvature, and even more preferably on the proxmial end of the lesser curvature around near or adjacent to the lower esophageal sphincter, provides superior results in the treatment of gastroesophageal reflux disease as compared to placement on the fundus, greater curvature, or antrum of the stomach or on or around the lower esophageal sphincter.

Although not wishing to be limited by theory, it is thought that these improvements are at least in part due to the greater concentration of nerve fibers in the region of the lesser curvature as well as less expansion and contraction of the stomach muscles in the region of the lesser curvature during digestion processes. The placement of the electrostimulation device in the area of the lesser curvature is an easier surgical laparoscopic procedure, especially as compared to placement on or around the lower esophageal sphincter for treatment of gastroesophageal reflux disease, due to the easier access.

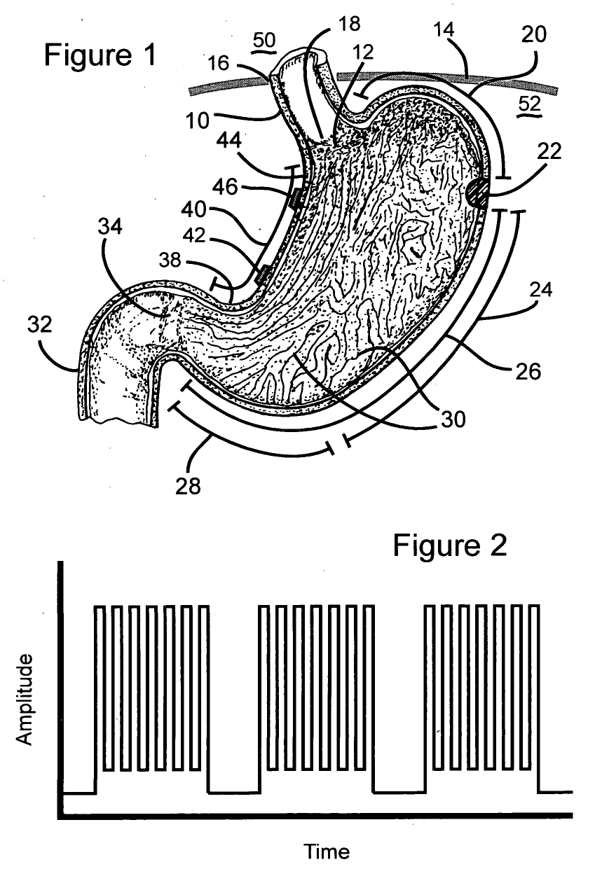

In order to further clarify the processes and devices for treating obesity and/or gastroesophageal reflux disease of a patient, according to the invention, the motor physiology of the gastric viscus is briefly described. As shown in FIG. 1, the stomach is supplied by the esophagus 10, and has the fundus ventriculi 20, the cardia 12, the body or corpus ventriculi 24, the antrum 28, the pylorus 34, the duodenum 32, and mucous folds or rugae 30. The esophagus 10 begins as a continuation of the pharynx and descends through the thorax 50 where it pierces the diaphragm 14 through the esophageal hiatus 16 to enter the abdominal cavity 52. Within the abdominal cavity 52, the esophagus 10 is connected to the stomach at its superior portion. The lower esophageal sphincter 18 is located in the inferior part of the esophagus near or at its junction with the stomach and is generally surrounded by the cardia 12. The stomach is generally divided into two parts as regards its motility: the fundus ventriculi 20, which has tonic wall movements, and the central part or corpus 24, which is characterized by phasic activity. Propulsive gastric movements begin at a point proximate to the greater curvature 26 which is not clearly identified anatomically and is termed “gastric pacemaker” 22. The gastric pacemaker 22 sends electrical pulses (depolarization potential) at a rate of approximately three times per minute which spread in an anterograde direction along the entire stomach in the form of waves which have a general sinusoidal shape.

The antrum 28 of the stomach has a continuous phasic activity which has the purpose of mixing the food which is present in the stomach. The passage of food into the duodenum 32 is the result of a motility coordinated among the antrum 28, pylorus 34, and duodenum 32. The gastric pacemaker 22 spontaneously and naturally generates sinusoidal waves along the entire stomach; these waves allow the antrum 28, in coordination with the pylorus 34 and duodenum 32, to allow food to pass into the subsequent portions of the alimentary canal (i.e., intestines).

Now that the known physiology of the gastric motility of a mammal, such as a human being, has been established, the process according to the invention consists in artificially altering, by means of sequential electrical pulses and for preset periods of time, the natural gastric motility of a patient and/or the time and manner of contraction of the lower esophageal and pyloric sphincters to prevent emptying or slow down gastric transit, to prevent duodenal acidification during interdigestive phases, and/or to prevent gastric reflux in the last portion of the esophagus. More particularly, the sequential electrical pulses are generated by electrical stimulators 42 or 46 which are applied by laparoscopic means to a portion of the seromuscular layer of the lesser curvature 40 of the stomach of the patient. In this manner, the electrical stimulus generates one or more sinusoidal waves which start in the lesser curvature 40 and add, more or less synchronously, with those which correspond to the natural electrical activity of the stomach when emptying procedures are activated in the stomach. Preferably, the electrical stimulator is placed on the distal end (i.e., at or near the angular notch 38) of the lesser curvature 40 for treatment of obesity or on the proximal end (i.e., at or near the lower esophageal sphincter 18) for treatment of gastroesophageal reflux disease. The electrical stimulator induces in the stomach a motor incoordination (so-called antral tachygastria) in order to slow down or even prevent gastric transit through the pylorus into the intestine located downstream and thus allow treatment of obesity related to hyperalimentation, to modulate fasting gastric hypermotility for the treatment of relapsing duodenal ulcer in anxious subjects, and/or to improve the functionality of the lower esophageal and/or pyloric sphincters in treating reflux esophagitis and gastropathy induced by duodenogastric reflux.

The electrical stimulator or electrocatheter, according to the motor phenomenon to be corrected (e.g., induction of antral tachygastria in obesity, modulation of gastric hypermotility in anxious subjects, increase in sphincter function in reflux disorders), has a purpose-specific and potentially patient-specific frequency, intensity, duration, and period of stimulation, in addition to having a specific gastric location (i.e., lesser curvature 40) for application of the electrostimulation according to the type of disorder. The stimulator can be programmed both for continuous stimulation and for “on demand” stimulation (i.e., at the onset of a particular electrical activity which can be detected by the stimulator itself through the electrocatheter (if modified to monitor electrical activity) or under the control of the patient or medical personnel).

FIG. 1 shows two electrical stimulator devices (i.e., 42 and 46) located along the lesser curvature 40. Generally, however, only one of these electrical stimulator devices will be used for a given patient depending on the condition to be treated. Thus, for example, if the treatment is intended to mainly treat or control obesity, electrical stimulator device 42, located at or near the distal end of the lesser curvature 40 near the angular notch 38, will be preferred. On the other hand, if the treatment is intended to mainly treat or control gastroesophageal reflux disease, generally electrical stimulator device 46, located at or near the proximal end 44 of the lesser curvature 40, will be preferred. For patients having need of treatment for both conditions, an electrical stimulator could be located along the middle portion of the lesser curvature 40 so as to effectively stimulate both the distal and proximal ends of the lesser curvature. Alternatively, both electrical stimulators 42 and 46 could be implanted and used concurrently or separately to control or treat both conditions.

The electrical stimulator 42 or 46, in order to allow to perform iatrogenic tachygastria, preferably has a preset operating frequency and period which may obviously vary according to the alteration of stomach motility to be obtained and/or to the pathological condition of the patient. Generally, the electrical stimulator 42 or 46 has an operating frequency of about 2 to about 15 pulses per minute. Preferably, the process of this invention employs stimulation of the lesser curvature at a rate of about 2 to about 14 pulses/minute with each pulse lasting about 0.5 to about 4 seconds such that there is a pause of about 3 to about 30 seconds between the pulses. The electrical discharge of each pulse can vary from approximately 1 to 15 volts for voltage-controlled stimulation and from 2 to 15 milliamperes for constant current stimulation. More preferably, the pulse rate is about 12 pulses/minute with each pulse lasting about 2 seconds with a pause of about 3 seconds between pulses. Preferably, the pulse amplitude is about 0.5 to about 15 milliamps. More preferable, each pulse consists of a train of micro-bursts with a frequency of about 5 to about 100 sec⁻¹. FIG. 2 generally illustrates a preferred microburst pulse train provided to the lesser curvature of the stomach.

The present invention generally uses conventional laparoscopic or minimally invasive surgical techniques to place the desired electrostimulation device 42 or 46 on, or adjacent to, the lesser curvature 40, and preferably on the distal portion of the lesser curvature (i.e., adjacent to the angular notch 38) for the treatment of obesity and preferably on the proximal end (i.e., at or near the lower esophageal sphincter 18) for treatment of gastroesophageal reflux disease. The methods of present invention do not require the penetration of the diaphragm 14 for placement or operation of electrostimulation device 42 or 46. Conventional electrostimulation devices may be used in the practice of this invention. Such devices include, for example, those described in U.S. Pat. No. 5,423,872 (Jun. 3, 1995) (an implantable gastric electrical stimulator at the antrum area of the stomach which generates sequential electrical pulses to stimulate the entire stomach, thereby artificially altering the natural gastric motility to prevent emptying or to slow down food transit through the stomach); U.S. Pat. No. 5,690,691 (Nov. 25, 1997) (a portable or implantable gastric pacemaker employing a number of electrodes along the greater curvature of the stomach for delivering phased electrical stimulation at different locations to accelerate or attenuate peristaltic movement in the GI tract); U.S. Pat. No. 5,836,994 (Nov. 17, 1998) (an implantable gastric stimulator which incorporates direct sensing of the intrinsic gastric electrical activity by one or more sensors of predetermined frequency bandwidth for application or cessation of stimulation based on the amount of sensed activity); U.S. Pat. No. 5,861,014 (Jan. 19, 1999) (an implantable gastric stimulator for sensing abnormal electrical activity of the gastrointestinal tract so as to provide electrical stimulation for a preset time period or for the duration of the abnormal electrical activity to treat gastric rhythm abnormalities); PCT Application Serial Number PCT/US98/10402 (filed May 21, 1998) and U.S. patent application Ser. No. 09/424,324 (filed Jan. 26, 2000) (implant device equipped with tines to help secure it in the appropriate location); U.S. Pat. No. 6,041,258 (Mar. 21, 2000) (electrostimulation device with improved handle for laparoscopic surgery); U.S. patent application Ser. No. 09/640,201 (filed Aug. 16, 2000) (electrostimulation device attachable to enteric or endo-abdominal tissue or viscera which is resistance to detachment); PCT Application Serial Number PCT/US00/09910 (filed Apr. 14, 2000; Attorney Docket Number 3581/006 PCT) entitled “Gastric Stimulator Apparatus and Method for Installing” based on U.S. Provisional Application Ser. Nos. 60/129,198 and 60/129,199 (both filed Apr. 14, 1999); PCT Application Serial Number PCT/US00/10154 (filed Apr. 14, 2000; Attorney Docket Number 3581/004 PCT) entitled “Gastric Stimulator Apparatus and Method for Use” based on U.S. Provisional Application Ser. Nos. 60/129,209 (filed Apr. 14, 1999) and 60/466,387 (filed Dec. 17, 1999); and U.S. Provisional Patent Application Ser. No. 60/235,660 (filed Sep. 26, 2000) entitled “Method and Apparatus for Intentional Impairment of Gastric Motility and/or Efficiency by Triggered Electrical Stimulation of the Gastric Tract with Respect to the Intrinsic Gastric Electrical Activity.” All of these patents, patent applications, provisional patent applications, and/or publications are hereby incorporated by reference.

Preferred electrostimulation devices include electrocatheters having an elongated body with a distal end having an electrostimulation lead or leads mounted on, or attached to, the stomach in the region of the lesser curvature and a proximal end for attachment to a pulse generator. The electrostimulation lead or leads are attached to a power source through, or with, the pulse generator. Such preferred electrostimulation devices are described in, for example, PCT Application Serial Number PCT/US98/10402 (filed May 21, 1998), U.S. patent application Ser. No. 09/424,324 (filed Jan. 26, 2000), and U.S. patent application Ser. No. 09/640,201 (filed Aug. 16, 2000).

Although the present invention is especially adapted for treatment of obesity and/or control of weight and gastroesophageal reflux disease, it may also be employed in treatment regimes involving other stomach-related disorders including, for example, relapsing peptic duodenal ulcer of anxious subjects, gastric peptic disorders induced by duodenogastric reflux, esophageal peptic disorders induced by gastroesophageal reflux, and the like.

The present methods can also be used in combination with electrostimulation of other parts of the gastrointestinal tract. For example, electrostimulation could be applied to the region of the lesser curvature as well as one or more location within the gastrointestinal tract. The sites of electrostimulation could be phased or non-phased in relation to one another.

The methods and electrostimulators used in the present invention are susceptible to numerous modifications and variations, all of which are within the scope of the present inventive concept. Furthermore, all the details may be replaced with technically equivalent elements. The materials employed, the shapes, and the dimensions of the specific electrostimulators may be varied according to the requirements.

We claim:

1. A method for treatment of gastroesophageal reflux disease in a patient, said method comprising
implanting an electrostimulation device comprising one or more electrostimulation leads and an electrical connector for attachment to a pulse generator such that the one or more electrostimulation leads are attached to, or adjacent to, lesser curvature of a patient's stomach, whereby electrical stimulation can be provided to the lesser curvature through the one or more electrostimulation leads; and
supplying electrical stimulation to the lesser curvature through the one or more electrostimulation leads.

2. The method of claim 1, wherein the one or more electrostimulation leads are attached to, or adjacent to, the lesser curvature at its upper end.

3. The method of claim 1, wherein the electrical stimulation supplied to the lesser curvature has an operating frequency of about 2 to about 15 pulses per minute.

4. The method of claim 2, wherein the electrical stimulation supplied to the lesser curvature at a rate of about 2 to about 15 pulses per minute.

5. The method of claim 3, wherein the rate of the electrical stimulation supplied to the lesser curvature is about 2 to about 14 pulses/minute with each pulse lasting about 0.5 to about 4 seconds such that there is a pause of about 3 to about 30 seconds between the pulses.

6. The method of claim 4, wherein the rate of the electrical stimulation supplied to the lesser curvature is about 2 to about 14 pulses/minute with each pulse lasting about 0.5 to about 4 seconds such that there is a pause of about 3 to about 30 seconds between the pulses.

7. The method of claim 3, wherein each pulse consists of a train of micro-bursts with a frequency of about 5 to about 100 sec⁻¹.

8. The method of claim 4, wherein each pulse consists of a train of L1 micro-bursts with a frequency of about 5 to about 100 sec⁻¹.

9. The method of claim 5, wherein each pulse consists of a train of micro-bursts with a frequency of about 5 to about 100 sec^{-1.}

10. The method of claim 6, wherein each pulse consists of a train of micro-bursts with a frequency of about 5 to about 100 sec⁻¹.

11. A method for treatment of gastroesophageal reflux disease in a patient said method comprising
implanting an electrostimulation device comprising an elongated body with a proximal and a distal end and having one or more electrostimulation leads and an electrical connector for attachment to a pulse generator at the proximal end such that the one or more electrostimulation leads are attached to, or adjacent to, lesser curvature of a patient's stomach, whereby electrical stimulation can be provided to the lesser curvature through the one or more electrostimulation leads and whereby, once the electrostimulation device is implanted, the one or more electrostimulation leads are at the distal end of the elongated body; and
supplying electrical stimulation to the lesser curvature through the one or more electrostimulation leads.

12. The method of claim 11, wherein the one or more electrostimulation leads are attached to, or adjacent to, the lesser curvature at its upper end.

13. The method of claim 11, wherein the electrical stimulation supplied to the lesser curvature has an operating frequency of about 2 to about 15 pulses per minute.

14. The method of claim 12, wherein the electrical stimulation supplied to the lesser curvature at a rate of about 2 to about 15 pulses per minute.

15. The method of claim 13, wherein the rate of the electrical stimulation supplied to the lesser curvature is about 2 to about 14 pulses/minute with each pulse lasting about 0.5 to about 4 seconds such that there is a pause of about 3 to about 30 seconds between the pulses.

16. The method of claim 14, wherein the rate of the electrical stimulation supplied to the lesser curvature is about 2 to about 14 pulses/minute with each pulse lasting about 0.5 to about 4 seconds such that there is a pause of about 3 to about 30 seconds between the pulses.

17. The method of claim 13, wherein each pulse consists of a train of micro-bursts with a frequency of about 5 to about 100 sec⁻¹.

18. The method of claim 14, wherein each pulse consists of a train of micro-bursts with a frequency of about 5 to about 100 sec⁻¹.

19. The method of claim 15, wherein each pulse consists of a train of micro-bursts with a frequency of about 5 to about 100 sec⁻¹.

20. The method of claim 16, wherein each pulse consists of a train of micro-bursts with a frequency of about 5 to about 100 sec⁻¹.

21. A method for treatment of obesity and gastroesophageal reflux disease in a patient, said method comprising
implanting at least one electrostimulation device comprising one or more electrostimulation leads and an electrical connector for attachment to a pulse generator such that the one or more electrostimulation leads are attached to, or adjacent to, lesser curvature of a patient's stomach, whereby electrical stimulation can be provided to the lesser curvature through the one or more electrostimulation leads; and
supplying electrical stimulation to the lesser curvature through the one or more electrostimulation leads.

22. The method as defined in claim 21, wherein at least one electrostimulation lead is attached to, or adjacent to, the lesser curvature at its upper end and at least one electrostimulation lead is attached to, or adjacent to, the lesser curvature at its lower end.
